(19) United States
(12) Patent Application Publication
(10) Pub. No.: US 2009/0253151 A1
(43) Pub. Date: Oct. 8, 2009

(54) Self-Renewing Master Adult Pluripotent Stem Cells

(75) Inventors: Satish Totey, Bangalore (IN); Jyothi Prasanna, Bangalore (IN)

Correspondence Address: OLIFF & BERRIDGE, PLC, P.O. BOX 320850, ALEXANDRIA, VA, 22320-4850, US

(73) Assignee: STEMPEUTICS RESEARCH PRIVATE LIMITED, BANGALORE (IN)

(21) Appl. No.: 12/310,546

(22) PCT Filed: Aug. 31, 2007
(86) PCT No.: PCT/IN2007/000384

§ 371 Date: May 15, 2009

(30) Foreign Application Priority Data
Sep. 1, 2006 (IN) 1588/CHE/2006

(51) Int. Cl. G01N 33/53; C12N 5/00; C12N 5/08; C12Q 1/02

(52) U.S. Cl. 435/7.21; 435/325; 435/371; 435/407; 435/29

(57) ABSTRACT

The present invention relates to a method for obtaining master adult pluripotent stem (MAPS) cells from adult human corneal epithelial tissues. The MAPS cells are obtained on the basis of pluripotent markers. Further the invention provides MAPS cells that are capable of self renewal and differentiation and have characteristics similar to that of human embryonic stem cells. The MAPS cells also retain the ability to differentiate into cells of different lineages. The composition comprising MAPS cells are useful for therapeutic purposes. Further, the invention provides a culture medium for proliferation of MAPS cells.

### FIELD OF INVENTION

The present invention relates to isolation purification and use of Master Adult Pluripotent Stem (MAPS) cells from adult human corneal epithelial tissues.

### BACKGROUND OF INVENTION

Cell or tissue regeneration is the process whereby multiple damaged bell types are replaced with new healthy cells to restore normal function of the tissue. Several theories have been proposed to explain the phenomenon of tissue or cell restoration in mammals or in the lower vertebrates. One of the theories suggests that the restoration of damaged tissue usually happen because of activation of stem cells normally present in the body or in particular organ.

Stem cells have the ability to divide without limit and to give rise to specialized cells. They are best described in the context of normal human development.

Human development begins when a sperm fertilizes an egg and creates a single cell that has the potential to form an entire human being, that cell is totipotent, meaning that its potential is total. After approximately four days, the totipotent cells enter the next stage of development called blastocysts. The blastocyst has an outer layer of cells called trophectoderm that gives rise to placenta. Inside there is a cluster of cells called the inner cell mass and that gives rise to all forms of tissue of the human body. Inner cell mass cells are pluripotent they can give rise to many, but not all, types of cells. Pluripotent stem cells undergo further specialization into stem cells that are committed to giving rise to cells with a particular function—for example, blood stem cells or skin stem cells.

Following the rule according to which, in ontogenesis, the younger/less differentiated the cell, the more pluripotent it is, it has been generally believed that embryonic stem cells are the only truly totipotent cells, whereas adult stem cells are capable of only maintaining the homeostasis of the tissue in which they belong. Embryonic stem cells are uncommitted. In the undifferentiated state these cells are expressed as alkaline phosphatase and surface antigens SSEA-3, SSEA-4, TRA-1-60, TRA-1-81 and transcription factors Oct-4 and Nanog. They show high telomerase activity, and show capabilities for extended self-renewal. Upon differentiation these cells express a wide variety of cell types) derived from ectodermal, mesodermal, and endodermal embryonic germ layers. Embryonic stem (ES) cells have been isolated from the blastocyst, inner cell mass or gonadal ridges of mouse, rabbit, rat, pig, sheep, primate and human embryos Evans and Kauffman, 1981; Iannaccone et al., 1994; Graves and Moreadith, 1993; Martin, 1981; Notarianni et al., 1991; Thomson, et al., 1995; Thomson, et al., 1998; Shamblott, et al., 1998, Heins, et al 2004).

It has been known for decades that stem cells with limited differentiation potential are present in post-natal tissues of mammals, and adult stem cells are already used clinically. For instance, hematopoietic stem cells can reestablish the hematopoietic system following myeloablation, and stem cells are being used to regenerate corneal and skin tissue. But recent studies report that adult tissues might contain cells with pluripotent characteristics.

Current concepts about tissue homeostasis and regeneration hold that another type of cells account for tissue regeneration and repair and regulate the supply of highly differentiated tissue-specific cells. These cells are called progenitor or blast cells and appear to be the least differentiated cells in a tissue.

Stem cells appear to possess great plasticity, but the cellular mechanisms regulating their behavior and fate are not understood. If these mechanisms can be harnessed to obtain cells specifically required for therapy, diagnosis or drug discovery, it may be possible to restore function to tissues and organ systems that have been compromised by congenital disorders, developmental malfunction, age, injury, and disease or drug exposure.

Undifferentiated stem cells with characteristics similar to that of embryonic stem cells have been isolated from postnatal mammals and these stem cells are isolated from brain (Jiang et al., 2002), bone marrow (Jiang et al., 2002; Reyes et al., 2002; Schwartz et al., 2002), blood (Zaho et al., 2003), skeletal muscle (Jiang et al., 2002, Young, 2004, Young and Black 2004, Young et al., 2004), dermis (Young 2004, Young and Black 2004), amniotic membrane (Miki et al., 2005), and inner ear (Li et al).

Pluripotent mesenchymal stem cells have been isolated from the skeletal muscle and are described in the U.S. Pat. No. 5,827,735 which is hereby incorporated by reference in its entirety.

Pluripotent stem cells were also isolated from the non-embryonic tissue selected from the group of muscle, dermis, fat, tendon, ligament, perichondrium, periosteum, heart, aorta, endocardium, myocardium, epicardium, large arteries and veins, granulation tissue, peripheral nerves, peripheral ganglia, spinal cord, dura, leptomeninges, trachea, esophagus, marrow, stomach, small intestine, large intestine, liver, spleen, pancreas, parietal peritoneum, visceral peritoneum, parietal pleura, visceral pleura, urinary bladder, gall bladder, kidney, associated connective tissues or bone marrow and are described in the US patent application no 20050255588 which is hereby incorporated by reference in its entirety.

Young et al., in U.S. Pat. No. 5,827,735 describes isolation of purified pluripotent mesenchymal stem cells. The cells were free from multinucleated myogenic lineage-committed cells and predominantly stellate shaped and isolated from skeletal muscle. However, these stem cells are good for wound healing process.

Young et al in US patent application no. 20050255588 describes the isolation of embryonic like pluripotent stem cells derived from the non-embryonic tissue selected from the group of muscle, dermis, fat, tendon, ligament, perichondrium, periosteum, heart, aorta, endocardium, myocardium, epicardium, large arteries and veins, granulation tissue, peripheral nerves, peripheral ganglia, spinal cord, dura, leptomeninges, trachea, esophagus, marrow, stomach, small intestine, large intestine, liver, spleen, pancreas, parietal peritoneum, visceral peritoneum, parietal pleura, visceral pleura, urinary bladder, gall bladder, kidney, associated connective tissues or bone marrow. The cells are self renewal and can differentiate into three major lineages such as ectoderm, mesoderm and endoderm.

Furcht et al in U.S. Pat. No. 7,015,037 describes the isolation of non-embryonic multipotent adult stem cells from bone marrow which have the potential to differentiate to form cells of a variety of cell lineages. However, multipotent stem cells are not pluripotent and have limited scope of differentiation.

Lucas et al, US patent application no. 20050260751 describes the isolation of pluripotent adult stem cells. These adult pluripotent stem cells have been isolated from skeletal muscle and express markers such as CD13, CD34, CD56, and CD117. However, it is known to those skilled in the art that CD34 markers are not explicitly used for isolating stem cells, rather they are specific for isolating hematopoetic lines. Therefore one cannot rule out the possibility of selection of stem cell due to contamination with blood cells present along with them. Stem cell expresses CD34 markers that are mainly hematopoetic in origin and have limited capacity to differentiate into various lineages and hence cannot be used for various applications and wide array of diseases.

Except as otherwise indicated, the disclosure of all patents, patent applications (and any patents which issue thereon, as well as any corresponding published foreign patent applications), and publications mentioned throughout this description are hereby incorporated by reference herein. It is expressly not admitted, however, that any of the documents incorporated by reference herein teach or disclose the present invention.

### SUMMARY OF INVENTION

Present invention relates to isolation and purification and use of Master Adult Pluripotent Stem (MAPS) cells from adult human corneal epithelial tissues. The invention further relates to method of obtaining MAPS cells, compositions comprising MAPS cells, a MAPS cell culture medium for proliferation of MAPS cells and use of the MAPS cells for transplantation.

The present invention relates to methods for obtaining Master Adult Pluripotent Stem (MAPS) cells from adult human corneal epithelial tissues. Further the invention provides MAPS cells that are capable of self renewal and differentiation and have characteristics similar to that of human embryonic stem cells. The MAPS cells also retain the ability to differentiate into cells of different lineages. The composition comprising MAPS cells is useful for therapeutic purposes. The invention also provides a culture medium for proliferation of MAPS cells.

The MAPS cells of the invention have similar characteristics to that of human embryonic stem cells. The invention also relates to uses of the stem cells for tissue engineering in cell or tissue transplantation and in gene therapy.

In one aspect, the invention relates to a method of obtaining master adult pluripotent stem (MAPS) cells from corneal epithelial tissues, comprising:

a. obtaining cells from the corneal epithelial tissues;

b. culturing the cells in a growth culture medium to obtain MAPS cells;

c. selecting MAPS cells based on the expression of pluripotent markers, and

d. culturing MAPS cells in a MAPS cell culture medium.

In another aspect, the invention relates to MAPS cells obtained from human adult corneal epithelial tissues, ensuring pluripotency in nature, having self renewal capacity and more plasticity.

In yet another aspect, the invention relates to a composition comprising MAPS cells for use as a therapeutic agent.

In another aspect, the invention relates to lineage cells derived from MAPS cells.

In yet another aspect, the invention relates to MAPS cell culture medium for proliferation of MAPS cells, wherein said culture medium comprises a basal culture medium and an effective amount of supplements.

### BRIEF DESCRIPTION OF DRAWINGS

FIG. 1: Isolated Master Adult Pluripotent Stem (MAPS) Cells and its morphology. Panel-A is unsorted cells and panel B is sorted MAPS cells showing high nuclear cytoplasmic ratio.

FIG. 2: Immunocytochemistry micrographs illustrating the expression of embryonic cell surface markers on MAPS cells following 10 passages. Shown are dark field images of MAPS cells labeled with monoclonal antibodies against SSEA-4, TRA-1-60, Vimentin (20 times magnification). Immunofluorescence analysis further confirms that MAPS cells are positive for SSEA-4 which is embryonic stem cell marker. Other stem cell marker is TRA-1-60.

FIG. 3: Presence of pluripotent embryonic stem cell markers on the MAPS cells using RT-PCR.

### DETAILED DESCRIPTION OF THE INVENTION

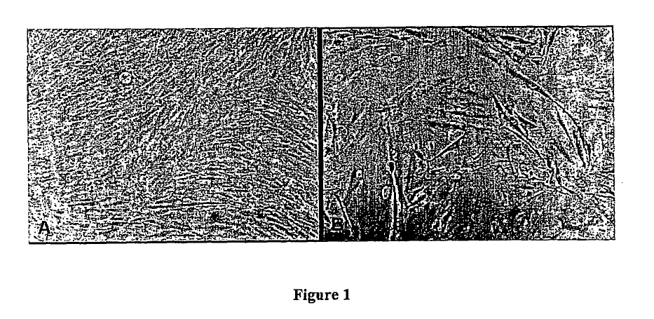

The present invention relates to isolation purification and use of master adult pluripotent stem (MAPS) cells from adult human corneal tissues. The MAPS cells of the invention have similar characteristics to that of human embryonic stem (hES) cells. MAPS cells isolated from human corneal epithelial tissues have distinct advantages in cell therapy such as, surgically easy accessibility, relatively higher proportion of pluripotent stem cells and further it makes the corneal epithelial tissue a potential tissue for therapy especially autografts thereby eliminating the problem of rejection. MAPS cells have high nuclear cytoplasmic ratio (See FIG. 1). The invention further relates to method of obtaining MAPS cells, compositions comprising MAPS cells, and a MAPS cell culture medium for proliferation of MAPS cells and use of MAPS cells for self renewal and differentiation into the cells of endodermal, ectodermal and mesodermal lineages.

In accordance with the invention, the Master Adult Pluripotent Stem (MAPS) Cells have use in both diagnostic and therapeutic purpose. These MAPS cells are uncommitted or undifferentiated stem cells and are pluripotent embryonic-like stem cells. This derives from the fact that these stem cells can be isolated from non-embryonic, postnatal human adult tissue and are capable of self renewal and differentiation into the cells of endodermal, ectodermal and mesodermal lineages. Thus, differentiated lineages any of the endodermal, ectodermal and mesodermal lineages can be provided from a single, self-renewable source of cells obtainable from human source even into and through adulthood. Further, MAPS cells can be stimulated by particular growth factors to proliferate. If activated to proliferate, MAPS cells have life-spans limited to 30-50 cell doublings before programmed cell senescence and death occurs.

The term used in the specification have the same meaning as used in the art, unless specifically stated otherwise. The term pluripotent stem cells, pluripotent embryonic-like stem cells, pluripotent mesenchymal stem cells, stem cells or master adult pluripotent stem cells, MAPS cells may be used herein interchangeably and as used through out the applications which are derived from non-embryonic tissue and capable of self-renewable and capable of differentiating into ectoderm, endoderm and mesoderm. These MAPS cells are pluripotent embryonic-like stem cells.

In one embodiment, the present invention relates to a method of obtaining master adult pluripotent stem (MAPS) cells from corneal epithelial tissues, said method comprising:
a. obtaining cells from the corneal epithelial tissues;
b. culturing the cells in a growth culture medium to obtain MAPS cells;
c. selecting MAPS cells based on the expression of pluripotent markers; and
d. culturing MAPS cells in a MAPS cell culture medium.

In another embodiment, the present invention relates to obtaining cells from the human corneal epithelial tissues.

In yet another embodiment, the present invention relates to the growth culture medium selected from a group consisting of Dulbecco's Modified Eagle's Medium-F-12: Nutrient mixture F-12 (Ham) (1:1) with supplements, DMEM-F-12 (1:1) with supplements, Dulbecco's Modified Eagle's Medium (DMEM) high glucose with supplements, Knockout Dulbecco's Modified Eagle's Medium (DMEM) with supplements and Dulbecco's Modified Eagle's Medium (DMEM) low glucose with supplements.

Still another embodiment of the present invention is directed to supplements added to the growth medium, which are selected from a group consisting of 1-10% fetal bovine serum, 1-10% knockout serum supplements, 1-10% Plasmalysate-A, 1-10% human serum, 1-10% calcium treated plasma, 1-5% human serum albumin, 1-5 ng/ml Epidermal Growth Factor (EGF), 0.5-5 μg/ml insulin, 0.5-5 μg/ml transferrin, 0.5-5 μg/ml sodium selenite, 1-10 μM Forskolin, 0.1-0.5 μg/ml hydrocortisone, 0.1-0.5% DMSO and a combination thereof.

Still yet another embodiment of the present invention provides MAPS cells selected by magnetic affinity cell sorting using specific antibodies.

In another embodiment, the present invention provides MAPS cells selected by fluorescent activated cell sorting using specific antibodies.

In yet another embodiment, the present invention provides specific antibodies against antigens used for selection of MAPS cell, wherein the antigens are selected from a group consisting of SH-1, SH-2, CD105, CD73, CD90, SSEA-4, SSEA-3, SSEA-1, CD44 and CD13.

In another embodiment, the present invention relates to selection of MAPS cells on the basis of positive expression of pluripotent marker, said pluripotent markets are selected from a group consisting of CD10, CD56 CD90, CD73, CD105, CD13, CD44, SSEA-4, SSEA-3, Oct-4, Sox-2, Nanog, Rex-1, Vimentin, TDGF1, TRA-1-60, TRA-1-81 and TERT.

In an additional embodiment, the present invention relates to selection of MAPS cells on the basis of negative expression of pluripotent marker, said pluripotent markers are selected from a group consisting of CD7, CD11, CD16, CD19, CD21, CD31, CD34, CD45, CD50, CD54, CD135, CD117, CD106, CD123, CD133, SSEA-1 and H LA-DR.

In still another embodiment, the present invention provides MAPS cell culture medium such as Dulbecco's Modified Eagle's Medium-F-12 (1:1) (DMEM-F-12 (1:1)) with supplements, Dulbecco's Modified Eagle's Medium (DMEM) high glucose with supplements, Knockout Dulbecco's Modified Eagle's Medium (DMEM) with supplements and Dulbecco's Modified Eagle's Medium (DMEM) low glucose with supplements.

In another embodiment, the present invention provides media with supplements, wherein the supplements added to the media are selected from 1-10% fetal bovine serum, 1-10% knockout serum supplements, 1-10% Plasmalysate-A, 1-10% human serum, 1-10% calcium treated plasma, 1-5% human serum albumin, 1-8 ng/ml Basic Fibroblast Growth Factor (bFGF), 1-10 ng/ml of Leukemia inhibitory factor (LIF), 1-5 ng/ml Epidermal Growth Factor (EGF), 0.5-5 μg/ml insulin, 0.5-5 μg/ml transferrin, 0.5-5 μg/ml sodium selenite, 0.1-0.5 μg/ml hydrocortisone and 0.1-0.5% Di-methyl sulphoxide (DMSO), activin A, activin B, PDGF, TGF-β and a combination thereof.

In yet another embodiment the present invention provides MAPS cells, said MAPS cells are capable of self renewal and differentiation.

In yet another embodiment, the present invention provides MAPS cell obtained from human corneal epithelial tissues, wherein said MAPS cell are capable of differentiation into cells of ectoderm, mesoderm and endoderm lineages.

In another embodiment, the present invention provides MAPS cells capable of differentiation into cells selected from a group consisting of cardiac cells, neuronal cells, hepatocytes, pancreatic beta cells, osteoblasts, myocytes, chondrocytes and adipocytes.

In yet another embodiment, the present invention provides MAPS cells obtained from corneal epithelial tissues, wherein said MAPS cell are useful for differentiation into cells of ectoderm, mesoderm and endoderm lineages.

In another embodiment, present invention provides MAPS cells obtained from corneal epithelial tissues, wherein the MAPS cell are useful for differentiation into cells, said cells are selected from a group consisting of cardiac cells, neuronal cells, hepatocytes, pancreatic beta cells, osteoblasts, chondrocytes and adipocytes.

In one embodiment, the present invention provides the human corneal epithelial tissues for obtaining MAPS cells.

In another embodiment, present invention provides MAPS cells that are P63 negative. The MAPS cells negative in P63 have been isolated for the first time. MAPS cells negative in P63 negative are uncommitted or undifferentiated stem cells and are pluripotent embryonic-like stem cells which are capable of self renewal and differentiation into the cells of endodermal, ectodermal and mesodermal lineages.

In another embodiment, present invention provides MAPS cells that are P63 negative and express SSEA-4, CD10, CD13, and CD56 antigens on these MAPS cells. These MAPS cells have been isolated for the first time. These MAPS cells are uncommitted or undifferentiated stem cells and are pluripotent embryonic-like stem cells which are capable of self renewal and differentiation into the cells of endodermal, ectodermal and mesodermal lineages.

In still another embodiment, the present invention provides a composition comprising MAPS cells.

In yet another embodiment, the present invention provides a composition comprising MAPS cells to be used as a therapeutic agent.

In one embodiment, the present invention provides a composition enriched with MAPS cells.

In still another embodiment, the present invention provides a therapeutic composition comprising MAPS cells.

In one embodiment, the present invention provides a composition comprising MAPS cells for treatment of cellular debilitation, derangement and/or dysfunction in mammals, where the composition further comprising a therapeutically effective amount of MAPS cells or a pharmaceutically acceptable medium or carrier.

In another embodiment, the present invention provides lineage cells derived from MAPS cells.

In still another embodiment, the present invention relates to MAPS cell culture medium for proliferation of MAPS cells, wherein said culture medium comprises

(a) a basal culture medium and

(b) an effective amount of supplements.

In another embodiment, the present invention relates to basal culture medium, wherein the culture medium is selected from a group consisting of Dulbecco's Modified Eagle's Medium-F-12 (1:1) (DMEM-F-12 (1:1)) with supplements, Dulbecco's Modified Eagle's Medium (DMEM) high glucose with supplements, Knockout Dulbecco's Modified Eagle's Medium (DMEM) with supplements and Dulbecco's Modified Eagle's Medium (DMEM) low glucose with supplements.

In yet another embodiment, the present invention relates to media with supplements, wherein the supplements added to the media are selected from a group consisting of 1-10% fetal bovine serum, 1-10% knockout serum supplements, 1-10% Plasmalysate-A, 1-10% human serum, 1-10% calcium treated plasma, 1-5% human serum albumin, 1-8 ng/ml Basic Fibroblast Growth Factor (bFGF), 1-10 ng/ml of Leukemia inhibitory factor (LIF), 1-5 ng/ml Epidermal Growth Factor (EGF), 0.5-5 μg/ml insulin, 0.5-5 μg/ml transferrin, 0.5-5 μg/ml sodium selenite, 0.1-0.5 μg/ml hydrocortisone and 0.1-0.5% Di-methyl sulphoxide (DMSO), activin A, activin B, PDGF, TGF-β and a combination thereof.

In another embodiment, the present invention provides use of MAPS cells for self renewal or differentiation.

In still another embodiment, the present invention provides use of MAPS cells for differentiating into cells of ectoderm, mesoderm and endoderm lineages.

In another embodiment, the present invention provides use of MAPS cells for differentiating into cells selected from a group consisting of cardiac cells, neuronal cells, hepatocytes, pancreatic beta cells, osteoblasts, chondrocytes and adipocytes.

In still another embodiment, the present invention provides use of MAPS cells for the manufacture of a medicament for use in the treatment of a disease selected from the group consisting of Parkinson's disease; Huntington's disease; motor neuron disease; heart disease; diabetes; liver disease (eg cirrhosis); renal disease; AIDS.

In another embodiment, the present invention provides use of MAPS cells for the manufacture of a medicament for tissue repair or transplantation in mammals.

In still another embodiment, the present invention provides use of MAPS cells in tissue engineering and gene therapy.

In still another embodiment, the present invention provides use of MAPS cells for the manufacturing of a medicament for the prevention of and/or treatment of cellular debilitations, derangements and/or dysfunctions and/or for transplantations and/or other disease states in mammals.

In yet another embodiment, the present invention provides a method of treatment of an animal, preferably a human, comprising administering a cell composition comprising MAPS cells which are induced to differentiate into at least one cell-type selected from a group consisting of cardiac cells, neuronal cells, hepatocytes, pancreatic beta cells, osteoblasts, chondrocytes and adipocytes.

In accordance with the present invention small biopsy of corneal epithelial tissue was removed surgically while carrying out cataract surgery after obtaining informed consent from the patient. Appropriate Institutional Ethics committee approval was taken before initiating this procedure. Indian Council of Medical Research was suitably informed and appropriate approval was taken as a regulatory requirement. All the human subjects were treated according to the Helsinki protocol. Biopsy was removed from superior or temporal quadrant of the corneal surface which was rich in corneal stem cells by lamellar keratectomy. Biopsy was then transported to the laboratory in an appropriate medium for further processing. Care was taken to process the biopsy immediately after surgical removal.

In accordance with the present invention, corneal epithelial cells were cultured and grown on Petri-dish bio-coated with either gelatin, matrigel, laminin, fibronectin or collagen or combination thereof in a medium such as DMEM-F-12 (1:1) or DMEM high glucose supplemented with 1-10% fetal bovine serum or 1-10% knockout serum supplements, 1-10% Plasmalysate-A, 1-10% human serum (preferably from blood group ‘O’ Rh+ve), 1-16% calcium treated plasma or 1-5% human serum albumin (HSA) in combination with several growth factors and culture supplements for at least 5 population doubling.

In accordance with the present invention the pluripotent embryonic-like stem cells were separated from the entire unsorted cells by the technique like magnetic affinity cell sorting (MACS) or florescent activated cell sorting (FACS) using specific antibodies against antigens such as SH-1 or SH-2 or SSEA-4. MAPS cells are isolated and re-plated on bio-coated Petri-dishes for further culture or further use.

In accordance with the present invention, sorted pluripotent embryonic-like stem cells were cultured and grown on Petri-dish bio-coated with either gelatin, matrigel, laminin, fibronectin or collagen or combination thereof in a medium such as DMEM-F-12 (1:1) or DMEM high glucose supplemented with 1-10% fetal bovine serum or 1-10% knockout serum supplements, 1-10% Plasmalysate-A, 1-10% human serum (preferably from blood group ‘O’ Rh+ve), 1-10% calcium treated plasma or 1-5% human serum albumin (HSA) in combination with several growth factors and culture supplements for faster growth.

In accordance with the present invention culture medium was supplemented with several growth factors and other culture supplements such as 1-8 ng/ml bFGF, 1-10 ng/ml LIF, 0.1-5 ng/ml EGF, 0.5-5 μg/ml insulin, 0.5-5 μg/ml transferrin, 0.5-5 μg/ml sodium selenite, 0.1-0.5 μg/ml hydrocortisone and 0.1-0.5% DMSO.

In accordance with the present invention the embryonic-like pluripotent undifferentiated stem cells were characterized for pluripotent markers such as Oct-4, Nanog, Rex-1, TDGF-1, Tert and Sox-2 using RT-PCR. Stem cells were also characterized for other markers such as SSEA-1, SSEA-3, SSEA-4, OCT-4, SOX-2, TRA-1-60, TRA-1-81, Alkaline phosphatase using immunocytochemistry. Cell surface antigens such as CD73, CD105, CD90, CD34, CD45, HLA-DR are also checked by flow cytometry analysis. In accordance with forgoing objects, the present invention provides the method of isolation and purification of Master Adult Pluripotent Stem (MAPS) Cells from non-embryonic tissue.

Further, the present invention extends to pluripotent embryonic like stem cells derived from post-natal tissue which is capable of self renewal and differentiation into ectoderm, mesoderm and endoderm lineages.

In addition the present invention extends to pluripotent embryonic like stem cells derived from adult human eye.

The present invention provides a method of isolation purification and use of Master Adult Pluripotent Stem (MAPS) Cells into several passages without loosing sternness of the pluripotent stem cells.

It is surprisingly found that the MAPS cells derived from adult human corneal epithelial tissue are capable of self renewal and differentiation. The MAPS cells have characteristics similar to that of human embryonic like stem cells. The MAPS cells have the ability to differentiate into cells of different lineages. The MAPS cells have the ability to differentiate into cells of ectoderm, mesoderm and endoderm lineages.

The present invention provides the method of isolation and purification of Master Adult Pluripotent Stem (MAPS) Cells into several passages into culture medium.

The present invention provides the method of isolation and purification of Master Adult Pluripotent Stem (MAPS) Cells, which are grown into the medium such as DMEM-F-12 (1:1) or DMEM high glucose in combination with serum and several growth factors.

The present invention provides the method of isolation and purification of Master Adult Pluripotent Stem (MAPS) Cells, which are grown into the medium such as DMEM-F-12 (1:1) or DMEM high glucose in combination with serum such as fetal bovine serum, plasma, human serum albumin or human serum or combination thereof.

The present invention provides the method of isolation and purification of Master Adult Pluripotent Stem (MAPS) Cells, which are grown into the medium such as DMEM-F-12 (1:1) or DMEM high glucose in combination with several growth factors such as bFGF, LIF, EGF, insulin, transferrin, sodium selenite, hydrocortisone or combinations thereof.

The present invention further provides the method of isolation and purification of Master Adult Pluripotent Stem (MAPS) Cells for culturing in the culture dish bio-coated with attachment factors.

The present invention further relates to the characterization of Master Adult Pluripotent Stem (MAPS) Cells using RNA isolation and reverse transcriptase-polymerase chain reaction (RT-PCR) for RNA expression using specific primers known in the art for the detection of markers.

The present invention further relates to the characterization of Master Adult Pluripotent Stem (MAPS) Cells using surface antigens and using immunofluorescence such as SSEA-1, SSEA-3, SSEA-4, TRA-1-60, TRA-1-81, Alkaline phosphatase and SOX-2.

The present invention relates to the characterization of Master Adult Pluripotent Stem (MAPS) Cells using surface antigens using flow cytometry analysis wherein such cells are positive for CD90, CD73, CD105 and C-kit.

The present invention further relates to the characterization of Master Adult Pluripotent Stem (MAPS) Cells using surface antigens using flow cytometry analysis wherein such cells are negative for CD34, CD45, CD133 and HLA-DR.

As suggested earlier and elaborated further on herein, the present invention contemplates the use of Master Adult Pluripotent Stem (MAPS) Cells, including cells or tissues derived there from, for instance, in pharmaceutical intervention, methods and therapy, cell-based therapies, gene therapy, various biological and cellular assays, isolation and assessment of proliferation or lineage-commitment factors, and in varied studies of development and cell differentiation.

As previously noted herein, the ability to regenerate most human tissues damaged or lost due to trauma or disease is substantially diminished in adults. Every year millions of people suffer tissue loss or end-stage organ failure. Tissue loss may result from acute injuries as well as surgical interventions, i.e., amputation, tissue degeneration, and surgical extirpations with respect to cancer, traumatic tissue injury, congenital malformations, vascular compromise, elective surgeries etc. Options such as tissue transplantation and surgical intervention are severely limited by a critical donor shortage and possible long term morbidity. Three general strategies for tissue engineering have been adopted for the creation of new tissue, (1) Isolated cells or cell substitutes applied to the area of tissue deficiency or compromise (2) cells placed on or within matrices, in either closed or open systems (3) tissue-inducing substances, that rely on growth factors (including proliferation factors or lineage-commitment factors) to regulate specific cells to a committed pattern of growth resulting in tissue regeneration, and methods to deliver these substances to their targets.

A wide variety of transplants, congenital malformations, elective surgeries, diseases, and genetic disorders have the potential for treatment with the Master Adult Pluripotent Stem (MAPS) Cells of the present invention, MAPS cells or tissues derived there from alone or in combination with proliferation factors, lineage-commitment factors, or genes or proteins of interest. Preferred treatment methods include the treatment of tissue loss where the object is to provide cells directly for transplantation whereupon the tissue can be regenerated in vivo, recreate the missing tissue in vitro and then provide the tissue, or providing sufficient numbers of cells suitable for transfection or transformation for ex vivo or in vivo gene therapy.

As described above, the Master Adult Pluripotent Stem (MAPS) Cells of the present invention have the unique capacity to differentiate into cells of any of the ectodermal, mesodermal and endodermal lineage. Thus, the Master Adult Pluripotent Stem (MAPS) Cells of the present invention may be utilized in transplantation, cell replacement therapy, tissue regeneration, gene therapy, organ replacement and cell therapies wherein cells, tissues, organs of mesodermal, ectodermal and/or endodermal origin are derived in vivo, ex vivo or in vitro. Endoderm cell, tissue or organ therapy and/or regeneration and/or therapy utilizing the Master Adult Pluripotent Stem (MAPS) Cells of the invention or their derived differentiated or progenitor cells may be useful as the cell source for epithelial linings of the corneal surface and gastrointestinal tract, the pharynx, esophagus, stomach, intestine and to many associated glands, including salivary glands, liver, pancreas and lungs. In particular and as non-limiting examples, liver transplantation and pancreas cell replacement for diabetes is thereby contemplated. Mesoderm cell, tissue or organ therapy and/or regeneration and/or therapy utilizing the Master Adult Pluripotent Stem (MAPS) Cells of the invention or their derived differentiated or progenitor cells may useful as the cell source for smooth muscular coats, connective tissues, and vessels associated with tissues and organs and for replacement/therapy of the cardiovascular system, heart, cardiac muscle, cardiac vessels, other vessels, blood cells, bone marrow, the skeleton, striated muscles, and the reproductive and excretory organs. Ectoderm cell, tissue or organ therapy and/or regeneration and/or therapy utilizing the Master Adult Pluripotent Stem (MAPS) Cells of the invention or their derived differentiated or progenitor cells may useful as the cell source for the epidermis (epidermal layer of the skin), the sense organs, and the entire nervous system, including brain, spinal cord, and all the outlying components of the nervous system. It is surprisingly found that the MAPS cells derived from adult human corneal epithelial tissues are negative for P63 marker and are capable of self renewal and differentiation. The MAPS cells have characteristics similar to that of human embryonic like stem cells. The MAPS cells have the ability to differentiate into cells of different lineages.

The MAPS cells obtained from the adult human corneal epithelial tissues do not express P63 antigen but express SSEA-4, CD10, CD13, and CD56 antigens on these MAPS cells. These MAPS cells have been isolated for the first time These MAPS cells are uncommitted or undifferentiated stem cells and are pluripotent embryonic-like stem cells which are capable of self renewal and differentiation into the cells of endodermal, ectodermal and mesodermal lineages.

A significant benefit of the Master Adult Pluripotent Stem (MAPS) Cells of the present invention are their potential for self-regeneration prior to commitment to any particular tissue lineage (ectodermal, endodermal or mesodermal) and then further proliferation once committed. These proliferative and differentiative attributes are very important and useful when limited amounts of appropriate cells and tissue are available for transplantation. The isolation of Master Adult Pluripotent Stem (MAPS) Cells as tissue source for transplantation therapies, that (a) can be isolated and sorted; (b) has unlimited proliferation capabilities while retaining pluripotency; (c) can be manipulated to commit to multiple separate tissue lineages; (d) is capable of incorporating into the existing tissue; and (e) can subsequently express the respective differentiated tissue type, may prove beneficial to therapies that maintain or increase the functional capacity and/or longevity of lost, damaged, or diseased tissues.

In a further embodiment, the present invention relates to certain therapeutic methods which would be based upon the activity of the Master Adult Pluripotent Stem (MAPS) Cells of the present invention, including cells or tissues derived there from, or upon agents or other drugs determined to act on any such cells or tissues, including proliferation factors and lineage-commitment factors. One exemplary therapeutic method, is associated with the prevention or modulation of the manifestations of conditions causally related to or following from the lack or insufficiency of cells of a particular lineage, and comprises administering the Master Adult Pluripotent Stem (MAPS) Cells of the present invention, including cells or tissues derived there from, either individually or in mixture with proliferation factors or lineage-commitment factors in an amount effective to prevent the development or progression of those conditions in the host.

Additionally the present invention includes therapeutic methods, including transplantation of the Master Adult Pluripotent Stem (MAPS) Cells of the present invention, including lineage-uncommitted populations of cells, lineage-committed populations of cells, tissues and organs derived there from, in treatment or alleviation of conditions, diseases, disorders, cellular debilitations or deficiencies which would benefit from such therapy. These methods include the replacement or replenishment of cells, tissues or organs. Such replacement or replenishment may be accomplished by transplantation of the Master Adult Pluripotent Stem (MAPS) Cells of the present invention or by transplantation of lineage-uncommitted populations of cells, lineage-committed populations of cells, tissues or organs derived there from.

The present invention includes a method of transplanting Master Adult Pluripotent Stem (MAPS) Cells in a host comprising the steps of introducing into the host Master Adult Pluripotent Stem (MAPS) Cells of the present invention.

Thus, the problem solved by the present invention is isolation of MAPS cells from adult human corneal epithelial tissues that are capable of self renewal and differentiation. The MAPS cells of the present invention being P63 negative and express SSEA-4, CD10, CD13, and CD56 on these MAPS cells due to which the MAPS cells have characteristics similar to that of human embryonic like stem cells. The MAPS cells have the ability to differentiate into cells of different lineages.

The present invention provides Master Adult Pluripotent Stem (MAPS) Cells isolated from post-natal human adult. Master Adult Pluripotent Stem (MAPS) Cells have been obtained in the present invention from corneal epithelial tissue (See FIG. 1 & Example 1). No specific age have been studied for the presence of Master Adult Pluripotent Stem (MAPS) Cells. However, in the present study biopsy is taken from post natal human adult ranging from 21 years to 70 years old and no difference has been found in proliferation capacity, cell numbers, morphology, renewable capacity, sternness, characteristics and differentiation capacity. Corneal epithelial tissue biopsy is then processed and cultured for suitable time in a medium composition as stated above.

The present invention is further directed to a method of processing and culturing of corneal epithelial tissue biopsy. Details of the method are provided in Example 2. Other methods known in the art can also be used.

The details of process of culturing of Master Adult Pluripotent Stem (MAPS) cells are provided in Example 3.

The present invention also relates to characterization of Master Adult Pluripotent Stem (MAPS) Cells on the basis of morphology, immunocytochemistry (Example 4), molecular markers by RT-PCR (See Example 5), and cell surface markers by FACS analysis (Example 6). Other methods known to persons skilled in the art can also be used. Master Adult Pluripotent Stem (MAPS) cells are characterized for early embryonic markers such as SSEA-1, SSEA-3, SSEA-4, TRA-1-60, TRA-1-81, and the mesenchymal marker vimentin (see example 4). The details of the antibody dilutions used for the characterization of MAPS cells (example 4) and results are provided in Table 1.

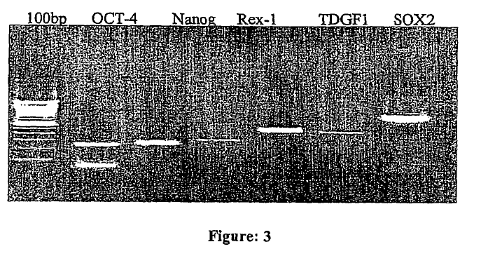

MAPS cells were also analyzed for the expression of pluripotent markers by RT-PCR. MAPS cells were positive for OCT-4, Nanog, Rex-1, TDGF, TERT, and SOX-2 (For details refer Example 5 and FIG. 3). This surprisingly showed that MAPS cells have embryonic like properties. Corneal stem cells are usually positive for P63 markers which is present in unsorted cells but absent in sorted cells. However, MAPS cells of the present invention are P63 negative. The MAPS cells negative in P63 have been isolated for the first time. The MAPS cells negative in P63 are uncommitted or undifferentiated stem cells and are pluripotent embryonic-like stem cells which are capable of self renewal and differentiation into the cells of endodermal, ectodermal and mesodermal lineages.

The details of the molecular markers for pluripotent cells and their results on MAPS cells using RT-PCR are provided in Table-2.

The MAPS cells were also analyzed for cell surface cluster differentiation (CD) markers to aid in isolating comparatively purified populations of these cells. For details refer Example 6. The details of the analysis of cell surface markers on MAPS cells are provided in Table 3.

The invention further relates to Karyotyping of the MAPS cells. Karyotyping was done after every 10 passages in order to determine the karyotypic instability. Details of the procedure followed are provided in Example 7. Other methods known in the art can also be used. It was observed that MAPS cells show karyotypic stability even up to 10 passages.

Telomerase Repeat Amplification Protocol (TRAP) was used for determining the telomerase activity. MAPS cells were harvested and washed once in Ca²⁺- and Mg²⁺-free phosphate buffered saline and then quickly frozen on dry ice before storage at −80° C. For details refer Example 8. It was found that MAPS cells have high telomerase activity and show ability for extended self-renewal.

Master Adult Pluripotent Stem (MAPS) Cells of the present invention have the capacity to differentiate into cells of any of the ectodermal, mesodermal and endodermal lineage. The capacity for such differentiation in vitro and in vivo, even to correct defects and function in vivo is demonstrated herein in Example 9.

### EXAMPLES

The following examples are intended to illustrate some of the embodiments of the present invention and should be construed as to limiting the present invention scope. The modifications and or additions to the different aspects of the present invention as described heretofore, is possible without departing from the field and scope of the present invention:

### Example 1

### Isolation of Master Adult Pluripotent Stem (MAPS) Cells

Master Adult Pluripotent Stem (MAPS) Cells have been isolated from post-natal human adult. Master Adult Pluripotent Stem (MAPS) Cells have been obtained in the present invention from corneal epithelial tissue (See FIG. 1).

Collection of Corneal Epithelial Tissue Biopsy

Small biopsy of corneal epithelial tissue is removed surgically while carrying out cataract surgery after obtaining informed consent from the patient. Appropriate Institutional Ethics committee approval is taken before initiating this procedure. Indian Council of Medical Research is suitably informed and appropriate approval is taken as a regulatory requirement. All the human subjects are treated according to the Helsinki protocol. Biopsy is removed from superior or temporal quadrant of the corneal surface which is rich in corneal stem cells by lamellar keratectomy. Biopsy is then transported to the laboratory in culture medium comprising of DMEM-F-12 (1:1) supplemented with 10% fetal calf serum or 10% knockout serum or 10% Plasmalysate-A (Baxter) or 10% human serum collected from the person having blood group ‘O’ and Rh+ve, or 5% human serum albumin (HSA), 0.25% dimethyl sulphoxide (DMSO), 2 ηg/ml recombinant human epidermal growth factor (rEGF), 5 μg/ml insulin, 5 μg/ml transferrin, 5 μg/ml sodium selenite, 0.5 μg/ml hydrocortisone, 10 μM Forskolin, gentamycin for further processing. Care is taken to process the biopsy immediately after surgical removal.

### Example 2

### Processing and Culture of Corneal Epithelial Tissue Biopsy

Tissue culture petri-dish is coated either with 1:3 diluted matrigel, 0.1% gelatin, 50-100 μg/ml fibronectin, 10-100 μg/ml laminin or 100 μg/ml collagen IV.

Corneal epithelial tissue biopsy is washed with medium as stated above and cut into small pieces. All the pieces are then arranged in a circular fashion with 20 μl medium. The medium composition is similar as stated above. Biopsies are cultured for 6 hours in CO₂ incubator at 37° C. and 5% CO₂ in air. After 6 hours of culture the biopsies are flooded with 1 ml of culture medium and allowed to grow for several days at 37° C. and 5% CO₂ in air. Once the cells become confluent, they are dissociated with either 0.05% trypsin-EDTA or dissociation buffer and re-plated on fresh bio-coated tissue culture dish at the ratio of 1:3. Cell are then expanded and serially passaged. Cells are serially passaged up to 5 passages.

### Example 3

### Culturing of Master Adult Pluripotent Stem Cells (MAPS)

After 5th passage, cells are again dissociated with 0.05% trypsin-EDTA. The cells are pipetted out into single cell suspension. Cells are counted using cell counter and re-suspended at the concentration of 1×10⁷ cells in 500 μl of buffer containing 0.5% serum, 2 mM EDTA and phosphate buffered saline (PBS). Cells are labeled with 10 μl of primary antibody against SSEA-4 or CD105 antigens. Cells are incubated for 20 minutes at 4-8° C. Cells are washed twice by adding 1-2 ml buffer and centrifuged at 1800 rpm for 10 minutes. Supernatant is removed completely and cells are re-suspended in 100 μl of buffer. Goat anti-mouse IgG micro-beads are added and again incubated for 20 minutes at 4-8° C. Cells are washed again by adding 2 ml of buffer and centrifuged at 1800 rpm for 10 minutes. Supernatant is removed and cells are re-suspended in 500 μl of buffer. MS column is placed in the magnetic field of a suitable MACS separator. Column is rinsed with 500 μl buffer and cells are loaded in the column. Unlabelled cells are separated out and collected by washing column with 500 μl of buffer three times. Column is then removed from the magnetic separator and placed into collection tube. 1 ml of buffer is loaded into the column and labeled cells are collected into the collection tubes. These cells are SSEA-4 positive or CD105 positive cells. Positive fraction of the cells are washed two times with culture medium and plated onto bio-coated plates as described earlier. SSEA-4 positive cells are cultured into medium comprises of DMEM-F-12 (1:1) or DMEM high glucose supplemented with 10% fetal bovine serum or 10% knockout serum or 10% plasmalysate-A or 10% human serum or 5% human serum albumin (HAS) or 10% calcium treated plasma. Culture medium is also supplemented with 0.5% DMSO, 5 μg/ml insulin, 5 μg/ml transferrin, 5 μg/ml sodium selenite, 2 ng/ml recombinant human epidermal growth factor, 0.5 μg/ml hydrocortisone, 4 ng/ml basic fibroblast growth factor, 10 ng/ml LIF and 200 μl Pen-strep. Cells are cultured in CO2 incubator at 37° C. and 5% Co2 in air. Cells usually get confluent by day 3-4 of initial plating.

After the cells getting confluent, the cells are dissociated with 0.05% trypsin-EDTA or dissociation buffer and re-plated on fresh bio-coated plate at the rate of 1:3 or 1:4. The cells are expanded and serially passaged.

### Example 4

### Characterization of Master Adult Pluripotent Stem (MAPS) Cells by Immunocytochemistry

Master Adult Pluripotent Stem (MAPS) Cells were characterized after every 5 passages on the basis of morphology, immunocytochemistry, molecular markers by RT-PCR, and cell surface markers by FACS analysis.

Looking at the morphology the Master adult pluripotent stem cells are slender, fibroblast like cells and look like similar to that of MAPS cells. Each cell has higher nuclear: cytoplasmic ratio and grow in adherent culture dish.

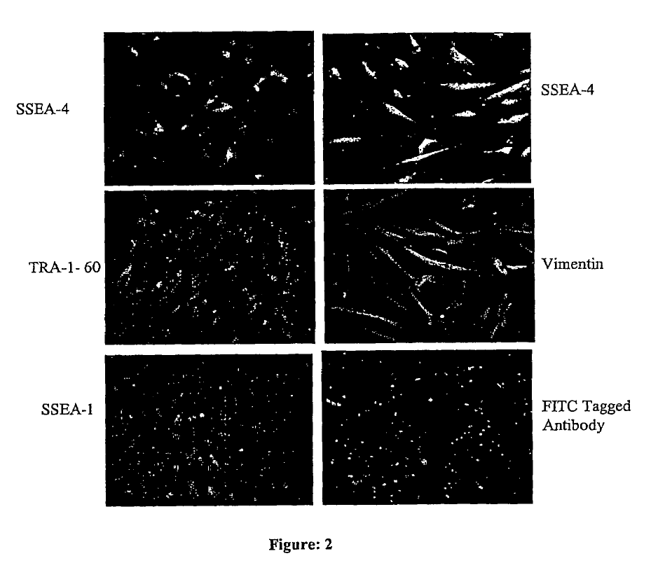

Master adult pluripotent stem cells (MAPS) are characterized for early embryonic markers such as SSEA-1, SSEA-3, SSEA-4, TRA-1-60, TRA-1-81 and the mesenchymal marker vimentin. Results clearly showed that MAPS cells express all the pluripotent cell surface markers such as SSEA-3, SSEA-4, TRA-1-60, TRA-1-81, Oct-4 whereas, SSEA-1 is not expressed on MAPS cells. These results are similar to that of human embryonic stem cells. It is known that SSEA-1 is only specific markers for mouse ES cells but not for human ES cells. On the other hand, SSEA-3 and SSEA-4 are not expressed on mouse ES cells (See FIG. 2).

MAPS cells were cultured on the two well chambered glass slides at 37° C. and 5% CO₂ in air till the cells get confluent. Culture media was carefully aspirated and cells were rinsed with phosphate buffered saline (PBS) without shaking the slides. 2% ice-cold paraformaldehyde fixative was added onto the slide and incubated for 3 minutes at room temperature. Immediately, cells were washed for 5 minutes with phosphate buffered saline. Blocking of the slide was performed with PBS containing 3% BSA to avoid any non-specific immunofluorescence and the cells were incubated in a sealed humidity chamber to prevent air-drying. Cells were washed three times for 5 minutes with phosphate buffered saline. Cells were then incubated with each prim antibodies separately diluted in PBS in 1% BSA for 5 hours at room temperature in the dark. Dilutions of the antibodies are given in Table-1. After incubation, cells were washed three times for 5 minutes with phosphate buffered saline. Cells were then incubated with fluorescein-conjugated secondary antibody in 1% BSA for 2 hours at room temperature in the dark. Cells were washed with phosphate buffered saline three times for 5 minutes again after completion of incubation. DAPI staining (net conc. 4 μg/ml) was performed for 5 minutes to stain the nucleus. Subsequently, cells were washed with PBS thrice for 5 minutes each. Cells were then mounted with cover slip and examined under microscope. Table 1 gives the results with various antibodies.

TABLE 1
Antibody dilutions used for the immunofluorescence analysis.

| Antibodies against the | | |
|---|---|---|
| following antigens | Dilution | Results |
| SSEA-1 | 1:100 | −ve |
| SSEA-3 | 1:100 | +ve |
| SSEA-4 | 1:100 | +ve |
| TRA-1-60 | 1:200 | +ve |
| TRA-1-81 | 1:200 | +ve |
| Vimentin | 1:500 | +ve |

### Example 5

### Characterization of Master Adult Pluripotent Stem (MAPS) Cells by RT-PCR

MAPS cells were also analyzed for the expression of pluripotent markers by RT-PCR. MAPS cells were positive for OCT-4, Nanog, Rex-1, TDGF, TERT, and SOX-2 (See FIG. 3). This clearly showed that MAPS cells have embryonic like properties. It is surprisingly found that MAPS cells have embryonic like properties and express pluripotent markers such as OCT-4, Nanog, Rex-1, TDGF, TERT, and SOX-2. Refer Table 2 for details. Corneal stem cells are usually positive for P63 markers which is present in unsorted cells but absent in sorted cells.

RNA extractions were carried out with the RNeasy mini kit. MAPS were vortexed for 1 min to shear genomic DNA before loading onto the columns, and then eluted in a minimum volume of 30 μl and a maximum volume of 2×50 μl RNAse-free water. RNA obtained with this procedure was essentially free of genomic DNA. When using different extraction procedures, a DNAse I treatment, followed by phenol extraction and ethanol precipitation, was applied to remove traces of contaminating DNA.

RNA obtained from the cells was reverse transcribed in the presence of 5 mM MgCl₂, 1×PCR Buffer II, 1 mM dNTPs, 25 u MuLV Reverse Transcriptase, 1 u RNA inhibitor, 2.5 μM Random hexamers in a final reaction volume of 20 μl. Reactions were carried out at 42° C. for 30 minutes in a thermocycler, followed by a 10 minute step at 99°, and then by cooling to 4° C.

2 μl of cDNA products were amplified with 1 unit of Taq polymerase in the buffer provided by the manufacturer which contains no MgCl₂, and in the presence of the specific primers well known in the art, used as an internal control. The amount of dNTPs carried over from the reverse transcription reaction is fully sufficient for further amplification. A first cycle of 10 minutes at 95° C., 45 seconds at 65° C. and 1 minute at 72° C. was followed by 45 seconds at 95° C., 45 seconds at 65° C. and 1 minute at 72° C. for 30 cycles. The conditions were chosen so that none of the RNAs analyzed reached a plateau at the end of the amplification protocol, i.e. they were in the exponential phase of amplification, and that the two sets of primers used in each reaction did not compete with each other. Each set of reactions always included a no-sample negative control.

The PCR products were loaded onto ethidium bromide stained 1 to 2% (depending on the size of the amplification products) agarose gels in TBE. A 100 bp DNA ladder molecular weight marker was run on every gel to confirm expected molecular weight of the amplification product.

Images of the RT-PCR ethidium bromide-stained agarose gels were acquired with a gel documentation system and quantification of the bands was performed. Band intensity was expressed as relative absorbance units. The ratio between the sample RNA to be determined and GAPDH or Actin was calculated to normalize for initial variations in sample concentration and as a control for reaction efficiency. Mean and standard deviation of all experiments performed were calculated after normalization to GAPDH or Actin.

It is surprisingly found that the MAPS cells have embryonic like properties. The MAPS cells have the ability to differentiate into cells of different lineages.

TABLE 2
Molecular markers for pluripotent cells and
their results on MAPS cells using RT-PCR

| Primers | Size | Results |
|---|---|---|
| OCT-4 | 572 | +ve |
| Nanog | 262 | +ve |
| Rex-1 | 303 | +ve |
| TDGF1 | 498 | +ve |
| SOX2 | 448 | +ve |
| P63 | 501 | −ve |
| TERT | 602 | +ve |
| GAPDH | 564 | +ve control |
| Connexin | 320 | +ve |
| 43 | | |
| Beta- | 353 | +ve control |
| actin | | |

### Example 6

### Immunophenotyping

Current invention is aimed at characterizing cell surface cluster differentiation (CD) markers on MAPS cells to aid in isolating comparatively purified populations of these cells. This study examined human pluripotent cells isolated from corneal epithelial tissue for the possible presence of CD markers. Flow cytometry showed cell populations positive for CD 90, CD 73, CD105, CD13, CD44, SSEA-4, SSEA-3, CD10 and CD56 and negative for CD34, CD45, HLA-DR, CD135, CD31, CD117, CD54, CD106 CD3, CD5, CD7, CD11b, CD14, CD15, CD16, CD19, CD25, CD45, CD65 and HLA-DR markers. Northern analysis revealed that CD13 and CD56 were actively transcribed at time of cell harvest. We report for the first time identification of CD10, CD13, and CD56 antigens on these MAPS cells. It is surprisingly found that MAPS cells derived from adult human corneal tissues express markers similar to human pluripotent cells. The isolation of MAPS cells from the human adult corneal tissue was carried for the first time and these MAPS cells are capable of self renewal and differentiation.

Aliquots of MAPS cells were allowed to expand at 37° C. and 95% air/5% CO2 humidified environment. After expansion, cells were dissociated with 0.05% trypsin-EDTA and re-suspended in buffer. The cells were then centrifuged and re-suspended in wash buffer at a concentration of 1×10⁶ cells/ml. Wash buffer consisted of phosphate buffer supplemented with 1% (v/v) FBS and 1% (w/v) sodium azide. Cell viability was >98% by the Trypan blue exclusion method. 100 μl of cell preparation 1×10⁵ were stained with saturating concentrations of fluorescein isothiocyanate-(FITC), phycoerythrin-(PE), conjugated CD7, CD10, CD11, CD13, CD16, CD19, CD21, CD31, CD34, CD44, CD45, CD50, CD54, CD73, CD90, CD105, CD106, CD117, CD123, CD133, CD135, SSEA-4, HLA-DR, and isotype matched controls. Briefly, cells were incubated in the dark for 30 min. at 4° C. After incubation, cells were washed three times with wash buffer and resuspended in 0.5 ml of wash buffer for analysis on the flow cytometer. Flow cytometry was performed on a LSR-II. Cells were identified by light scatter. Logarithmic fluorescence was evaluated (4 decade, 1024 channel scale) on 10,000 gated events. Analysis was performed using FACS DIVA and FLOW JO software and the presence or absence of each antigen was determined by comparison to the appropriate isotype control. An antigenic event was observed when the fluorescence was greater than 25% above its isotype control. Statistical analysis was performed on the pooled flow cytometric data from the three MAPS cell lines. Thus, a sample size of three was used for each CD marker. A mean value above 1000 cells was considered positive for any CD marker.

TABLE 3
Analysis of cell surface markers on MAPS cells

| Cell Surface | |
|---|---|
| Markers | Results |
| CD7 | −ve |
| CD10 | +ve |
| CD11 | −ve |
| CD13 | +ve |
| CD16 | −ve |
| CD19 | −ve |
| CD21 | −ve |
| CD31 | −ve |
| CD34 | −ve |
| CD44 | +ve |
| CD45 | −ve |
| CD50 | −ve |
| CD56 | +ve |
| CD73 | +ve |
| CD90 | +ve |
| CD105 | +ve |
| CD106 | −ve |
| CD 117 | −ve |
| CD123 | −ve |
| CD133 | −ve |
| CD135 | −ve |
| SSEA4 | +ve |
| HLA-DR | −ve |

Table 3 shows the results with various cell surface markers. We observed positive expression of markers, namely, CD10, CD13, CD44, CD56, CD73, CD90, CD105 and SSEA4. This clearly indicates that MAPS cells are similar to human embryonic cells.

### Example 7

### Karyotyping

It has been reported that karyotype instability can sometimes be observed with long-term passages of MAPS cells. In order to determine the karyotypic instability, karyotyping of the MAPS cells was done after every 10 passages. MAPS cells were grown in 60 mm plate on high density. Colcemid solution was added on the following day directly into the plate at the final concentration of 0.02 μg/ml. Cells were incubated for 2 hours at 37° C. and 5% CO₂. Culture media containing colcemid was removed after the incubation: was over and cells were dissociated with 0.05% trypsin free from EDTA. Cells were transferred into 15 ml tube and 10 ml FBS in DMEM-F-12 was added. Cells were washed by centrifuging at 1000 rpm for 5 minutes at room temperature. Supernatant was removed and re-suspend the pellet in 2 ml of warm hypotonic solution. Cells were mixed properly and incubated in a water bath at 37° C. for 30 minutes. 0.5 ml of fixative is added drop-wise with swirling. Cells were centrifuged again at 1000 rpm for 5 minutes at room temperature. Supernatant was aspirated and 1 ml of fixative was added drop-wise while swirling the cells. This was done at least 2 times.

To make the spread, surface of the slide is humidified by application of warm breath whilst holding the slide at a 45° angle. One drop of the suspended cells is carefully dropped from the height of approximately 0.5 meter using Pasteur pipette onto the top surface of the slide and it was allowed to air dry.

Slide was stained with freshly made Leishman's stain for 8 minutes and was rinsed in running water for 1 minute and air dried. Cells were mounted with coverslip using depex and karyotyping of the cells was undertaken. It was found that the MAPS cells show karyotypic stability even up to 10 passages.

### Example 8

### Telomerase Activity

For telomerase activity Telomerase Repeat Amplification Protocol (TRAP) was used. MAPS cells were harvested and washed once in Ca²⁺- and Mg²⁺-free phosphate buffered saline and then quickly frozen on dry ice before storage at −80° C. At the start of the test, the cell pellate were thawed and lysed for 30 minutes on ice by resuspension in TRAP buffer which contains 200 mM Tris-HCL, pH 8.3, 15 mM MgCl₂, 650 mM KCl, 0.5% Tween-20, 1 mg/ml bovine serum albumin and 10 mM EGTA in 1% Tween-20. The lysate from 1000 or 5000 cells equivalents was added to the master mix (1×TRAP buffer containing 50 μM deoxynucleoside triphosphate, 1 μg ACX, 1 μg U2, 0.1 mmol TSU2, 400 μM Cy5-TS primer and 2 U Taq polymerase. The final volume of the reaction mixture was 50 μl.

In parallel, as a control for false positive results, lysate from 1000 cells was heated at 85° C. for 10 minutes to inactivate endogenous telomerase and added to another 50 μl aliquot of the PCR master mix. Analysis of lysis buffer and lysate from equivalent number of 300 cells (telomerase positive E1 transformed cell line) were included as negative and positive controls, respectively. Thermocycler conditions for amplification were 30 minutes at 30° C. for telomerase repeat extension, followed by 31 cycles at 94° C. for 30 seconds and 60° C. for 30 seconds for amplification. Reaction products were separated on non-denaturing 12.5% polyacrylamide gels and visualized by phosphorimage and scanning for red fluorescence. Analysis of telomerase activity reveals that the MAPS cells have high telomerase activity, and show capabilities for extended self-renewal.

### Example 9

### a) Chondrogenic Differentiation

Differentiation of MAPS cells was induced in pellet culture in the medium DMEM-low glucose under serum free condition. Media was supplemented with 5 μg per milliliter Insulin, 5 μg per milliliter Transferrin, 5 ηg per milliliter Selenite and 10 ηg per milliliter transforming growth factor-beta-1. Media was changed every 3 days and cells were cultured for 3-4 weeks. On termination of cultures cells were stained with Toludin blue or safranin for analysis the differentiation potential.

### b) Osteogenic Differentiation

MAPS cells were plated at low density on tissue-culture-treated dishes in the presence of DMEM-low glucose medium supplemented with 10% fetal bovine serum, 50 μg per milliliter ascorbate-2 phosphate, 10⁻⁸ M dexamethasone, and 10 mM b-glycerol phosphate for 3-4 weeks Differentiated cells were stained with Von Kossa stain for analysis of osteogenic differentiation.

### c) Myogenic Differentiation

Confluent MAPS cells were maintained for 2-3 wk in α-MEM medium with 20% heat-inactivated FBS. More rapid induction was observed in the presence of medium conditioned for 24 h by differentiated C2C12 cells. Coculture of MAPS cells and C2C12 cells was carried out in α-MEM with 3% horse serum and 1% FBS.

### d) Adipogenic Differentiation

MAPS cells were grown to confluence in DMEM-low glucose medium supplemented with 10% fetal bovine serum. Medium was further supplemented with 50 μg per milliliter ascorbate-2 phosphate, 10⁻⁷ M dexamethasone and 50 μg per milliliter indomethacin. Cells were incubated for 3-4 weeks and stained with oil red 0 for analysis of differentiation into adipocytes.

MAPS cells are thus capable of differentiation into cells of different lineages such as cardiac cells, neuronal cells, hepatocytes, pancreatic beta cells, osteoblasts, myocytes, chondrocytes and adipocytes.

### REFERENCES

1. Evans M J, Kaufman M H, Establishment in culture of pluripotential cells from mouse embryos, Nature, July 9; 292(5819):154-6, 1981.
2. Graves K H, Moreadith R W, Derivation and characterization of putative pluripotential embryonic stem cells from preimplantation rabbit embryos, Mol Reprod Dev., December; 36(4):424-33, 1993.
3. Heins N, Englund M C, Sjoblom C, Dahl U, Torning A, Bergh C, Lindahl A, Hanson C, Semb H. Derivation, Characterization and Differentiation of human embryonic stem cells, Stem Cells, 22(3):367-76, 2004.
4. Iannaccone P M, Taborn G U, Garton R L, Caplice M D, Brenin D R, Pluripotent embryonic stem cells from the rat are capable of producing chimeras, Dev Biol. May; 163(1):288-92, 1994.
5. Jiang Y, Jahagirdar B N, Reinhardt R L, Schwartz R E, Keene C D, Ortiz-Gonzalez X R, Reyes M, Lenvik T, Lund T, Blackstad M, Du J, Aldrich S, Lisberg A, Low W C, Largaespada D A, Verfaillie C M, Pluripotency of mesenchymal stem cells derived from adult marrow, Nature. 2002 Jul. 4; 418(6893):41-9. Epub 2002 Jun. 20.
6. Li H, Liu H, Heller S, Pluripotent stem cells from the adult mouse inner ear, Nat. Med. 2003 October; 9(10):1293-9. Epub 2003 Aug. 31.
7. Martin G R, Isolation of a pluripotent cell line from early mouse embryos cultured in medium conditioned by teratocarcinoma stem cells, Proc Natl Acad Sci USA, December; 78(12):7634-8, 1981.
8. Miki T, Lehmann T, Cai H, Stolz D B, Strom S C, Stem cell characteristics of amniotic epithelial cells, Stem Cells, 2005 November-December; 23(10):1549-59. Epub 2005 Aug. 4, 2005.
9. Notarianni E, Galli C, Laurie S, Moor R M, Evans M J, Derivation of pluripotent, embryonic cell lines from the pig and sheep, Department of Genetics, University of Cambridge, UK. J Reprod Fertil Suppl.; 43:255-60, 1991.
10. Reyes M, Dudek A, Jahagirdar B. Koodie L, Marker P H, Verfaillie C M. Origin of endothelial progenitors in human postnatal bone marrow. J Clin Invest, February; 109(3):337-46, 2002.
11. Schwartz R E, Reyes M, Koodie L, Jiang Y, Blackstad M, Lund T, Lenyik T, Johnson S, Multipotent adult progenitor cells from bone marrow differentiate into functional hepatocyte-like cells, Clin Invest, May; 109(10):1291-302, 2002.
12. Hu W S, Verfaillie C M, Stem Cell Institute, University of Minnesota, Minneapolis 55455, USA.
13. Shamblott M J, Axelman J, Wang S, Bugg E M, Littlefield J W, Donovan P J, Blumenthal P D, Huggins G R, Gearhart J D, Derivation of pluripotent stem cells from cultured human primordial germ cells, Proc Natl Acad Sci USA, 1998 Nov. 10; 95(23):13726-31. Erratum in: Proc Natl Acad Sci USA 1999 Feb. 2; 96(3):1162.
14. Thomson J A, Kalishman J, Golos T G, Durning M, Harris C P, Becker R A, Hearn J P, Isolation of a primate embryonic stem cell line, Proc Natl Acad Sci USA, August 15; 92(17):7844-8, 1995.
15. Thomson, J. A., Itskovitz-Eldor, J., Shapiro, S., Waknitz, M. A., Sweirgiel, J. J., Marshall, V. S., et al, Embryonic stem cell lines derived from human blastocysts. Science, 282, 1145-114, 1998.
16. Young H E, Duplaa C, Yost M J, Henson N L, Floyd J A, Detmer K, Thompson A J, Powell S W, Gamblin T C, Kizziah K, Holland B J, Boev A, Van De Water J M, Godbee D C, Jackson S, Rimando M, Edwards C R, Wu E, Cawley C, Edwards P D, Macgregor A, Bozof R, Thompson T M, Petro G J Jr, Shelton H M, McCampbell B L, Mills J C, Flynt F L, Steele T A, Kearney M, Kirincich-Greathead A, Hardy W, Young P R, Amin A V, Williams R S, Horton M M, McGuinn S, Hawkins K C, Ericson K, Terracio L, Moreau C, Hixson D, Tobin B W, Hudson J, Bowyer F P 3rd, Black A C Jr, Clonogenic analysis reveals reserve stem cells in postnatal mammals, II. Pluripotent epiblastic-like stem cells, Anat Rec A Discov Mol Cell Evol Biol, March; 277(1):178-203, 2004.
17. Young H E, Duplaa C, Romero-Ramos M, Chesselet M F, Vourc'h P, Yost M J, Ericson K, Terracio L, Asahara T, Masuda H, Tamura-Ninomiya S, Detmer K, Bray R A, Steele T A, Hixson D, el-Kalay M, Tobin B W, Russ R D, Horst M N, Floyd J A; Henson N L, Hawkins K C, Groom J, Parikh A, Blake L, Bland L J, Thompson A J, Kirincich A, Moreau C, Hudson J, Bowyer F P 3rd, Lin T J, Black A C Jr, Adult reserve stem sells and their potential for tissue engineering, Cell Biochem Biophys, 2004; 40(1):1-80. Review.
18. Young H E, Black A C Jr, Adult stem cells, Anat Rec A Discov Mol Cell Evol Biol. 2004 January; 276(1):75-102. Review.
19. Young H E, Existence of reserve quiescent stem cells in adults, from amphibians to humans, Curr Top Microbiol Immunol, 2004; 280:71-109. Review.
20. Biopsies were obtained based on regulations and ethical guidelines set up by WORLD MEDICAL ASSOCIATION DECLARATION OF HELSINKI ETHICAL PRINCIPLES FOR MEDICAL RESEARCH INVOLVING HUMAN SUBJECTS

Adopted by the 18th WMA General Assembly Helsinki, Finland, June 1964 and amended by the

29th WMA General Assembly, Tokyo, Japan, October 1975

35th WMA General Assembly, Venice, Italy, October 1983

41st WMA General Assembly, Hong Kong, September 1989

48th WMA General Assembly, Somerset West, Republic of South Africa, October 1996

52nd WMA General Assembly, Edinburgh, Scotland, October 2000.

1-28. (canceled)

29. Master Adult Pluripotent Stem (MAPS) cells obtained from corneal epithelial tissues, wherein said MAPS cells are capable of self renewal and differentiation.

30. The MAPS cells as claimed in claim 29, wherein the MAPS cells are negative for CD31, CD117 and p63.

31. The MAPS cells as claimed in claim 29, wherein the MAPS cells are capable of differentiation into cells of ectoderm, mesoderm and endoderm lineages.

32. The MAPS cells as claimed in claim 29, wherein the MAPS cells are capable of differentiation into cells selected from a group comprising cardiac cells, neuronal cells, hepatocytes, pancreatic beta cells, osteoblasts, chondrocytes and adipocytes.

33. The MAPS cells as claimed in claim 29, wherein the corneal epithelial tissues are human tissues.

34. A composition comprising MAPS cells obtained from corneal epithelial tissues as claimed in claim 29, wherein the composition is used as a therapeutic agent.

35. A culture medium for proliferation of MAPS cells, wherein said culture medium comprises:
a. basal culture medium; and
b. an effective amount of supplements.

36. The culture medium as claimed in claim 35, wherein the basal culture medium is selected from a group comprising Dulbecco's Modified Eagle's Medium-F-12 (1:1) (DMEM-F-12 (1:1)) with supplements, Dulbecco's Modified Eagle's Medium (DMEM) high glucose with supplements, Knockout Dulbecco's Modified Eagle's Medium (DMEM) with supplements and Dulbecco's Modified Eagle's Medium (DMEM) low glucose with supplements, wherein the supplements are selected from a group comprising 1-10% fetal bovine serum, 1-10% knockout serum supplements, 1-10% Plasmalysate-A, 1-10% human serum, 1-10% calcium treated plasma, 1-5% human serum albumin, 1-8 ng/ml Basic Fibroblast Growth Factor (bFGF), 1-10 ng/ml of Leukemia inhibitory factor (LIF), 1-5 ng/ml Epidermal Growth Factor (EGF), 0.5-5 μg/ml insulin, 0.5-5 μg/ml transferrin, 0.5-5 μg/ml sodium selenite, 0.1-0.5 μg/ml hydrocortisone and 0.1-0.5% Di-methyl sulphoxide (DMSO), activin A, activin B, PDGF and TGF-β or a combination thereof.

37. A method of obtaining Master Adult Pluripotent Stem (MAPS) cells from corneal epithelial tissues, said method comprising steps of:
a. obtaining cells from the corneal epithelial tissues;
b. culturing the cells of step (a) in a growth culture medium to obtain MAPS cells;
c. selecting MAPS cells from step (b) based on the expression of pluripotent markers; and
d. culturing MAPS cells of step (c) in a MAPS cell culture medium.

38. The method as claimed in claim 37, wherein the MAPS cells are negative for CD31, CD117 and p63.

39. The method as claimed in claim 37, wherein the corneal epithelial tissues are human tissues.

40. The method as claimed in claim 37, wherein the growth culture medium is selected from a group consisting of Dulbecco's Modified Eagle's Medium-F-12: Nutrient mixture F-12 (Ham) (1:1) with supplements, DMEM-F-12 (1:1) with supplements, Dulbecco's Modified Eagle's Medium (DMEM) high glucose with supplements, Knockout Dulbecco's Modified Eagle's Medium (DMEM) with supplements and Dulbecco's Modified Eagle's Medium (DMEM) low glucose with supplements, wherein the supplements are selected from a group consisting of 1-10% fetal bovine serum, 1-10% knockout serum supplements, 1-10% Plasmalysate-A, 1-10% human serum, 1-10% calcium treated plasma, 1-5% human serum albumin, 1-5 ng/ml Epidermal Growth Factor (EGF), 0.5-5 μg/ml insulin, 0.5-5 μg/ml transferrin, 0.5-5 μg/ml sodium selenite, 1-10M Forskolin, 1 0.1-0.5 μg/ml hydrocortisone, 0.1-0.5% DMSO and a combination thereof.

41. The method as claimed in claim 37, wherein the MAPS cells are selected by magnetic affinity cell sorting or fluorescent activated cell sorting using specific antibodies against antigens selected from a group comprising SH-I, SH-2, CD105, CD73, CD90, SSEA-4, SSEA-3, SSEA-I, CD44 and CD13.

42. The method as claimed in claim 37, wherein the MAPS cells are selected based on the positive expression of pluripotent markers selected from a group comprising CD10, CD56 CD90, CD73, CD105, CD13, CD44, SSEA-4, SSEA-3, Oct-4, Sox-2, Nanog, Rex-1, Vimentin, TDGF1, TRA-1-60, TRA-1-81 and TERT and negative expression of pluripotent markers selected from a group comprising CD7, CDI1, CD16, CD19, CD21, CD31, CD34, CD45, CD50, CD54, CD135, CD117, CD106, CD123, CD133, S SE A-1 and HLA-OR.

43. The method as claimed in claim 37, wherein the MAPS cell culture medium is selected from a group consisting of Dulbecco's Modified Eagle's Medium-F-12 (1:1) (DMEM-F-12 (1:1)) with supplements, Dulbecco's Modified Eagle's Medium (DMEM) high glucose with supplements, Knockout Dulbecco's Modified Eagle's Medium (DMEM) with supplements and Dulbecco's Modified Eagle's Medium (DMEM) low glucose with supplements, wherein the supplements are selected from a group consisting of 1-10% fetal bovine serum, 1-10% knockout serum supplements, 1-10% Plasmalysate-A, 1-10% human serum, 1-10% calcium treated plasma, 1-5% human serum albumin, 1-8 ng/ml Basic Fibroblast Growth Factor (bFGF), 1-10 ng/ml of Leukemia inhibitory factor (LIF), 1-5 ng/ml Epidermal Growth Factor (EGF), 0.5-5 μg/ml insulin, 0.5-5 μg/ml transferrin, 0.5-5 μg/ml sodium selenite, 0.1-0.5 μg/ml hydrocortisone and 0.1-0.5% Di-methyl sulphoxide (DMSO), activin, ′A, activin B, PDGF, TGF-β and a combination thereof.
